(19) United States
(12) Patent Application Publication
(10) Pub. No.: US 2026/0237467 A1
(43) Pub. Date: Aug. 13, 2026

(54) TECHNOLOGIES FOR INDIVIDUALIZED METAGENOMIC PROFILING

(71) Applicant: BATTELLE MEMORIAL INSTITUTE, Columbus, OH (US)

(72) Inventors: Carrie HOWLAND, Columbus, OH (US); Craig M. BARTLING, Columbus, OH (US); Bryan GEMLER, Columbus, OH (US); Patrick FULLERTON, Columbus, OH (US); Jared SCHUETTER, Columbus, OH (US); Sayak MUKHERJEE, Columbus, OH (US); Rachel R. SPURBECK, Columbus, OH (US)

(21) Appl. No.: 19/154,682

(22) PCT Filed: Feb. 8, 2024
(86) PCT No.: PCT/US2024/014945

§ 371 Date: Aug. 7, 2025

Related U.S. Application Data
(60) Provisional application No. 63/444,104, filed on Feb. 8, 2023.

(51) Int. Cl. G16B 45/00; C12Q 1/6806; C12Q 1/6886; G16B 20/00; G16B 30/00; G16B 40/20; G16H 50/20

(52) U.S. Cl. CPC G16B 45/00; C12Q 1/6806; C12Q 1/6886; G16B 20/00; G16B 30/00; G16B 40/20; G16H 50/20; C12Q 2600/118; C12Q 2600/154

(57) ABSTRACT

Technologies for individualized metagenomics profiling include a computing device that may be in communication with multiple client devices. The technologies include receiving a genome sequence for an individual, mapping the genome sequence to generate a genome map compared to a predetermined sample human genome, mapping one or more chimeric sequences associated with an identified pathogen to the genome sequence to generate a chimera map, and mapping one or more active transposons to the genome sequence to generate a transposon map. The technologies further include generating a biomedical fingerprint associated with the individual by integrating the genome map, the chimera map, and the transposon map. The technologies may include mapping an epigenetic profile to the genome sequence to generate an epigenetic map, and generating the biomedical fingerprint further comprises overlaying the genome map, the chimera map, the transposon map, and the epigenetic map. Other embodiments are described and claimed.

### CROSS-REFERENCE TO RELATED APPLICATIONS

This application claims the benefit of and priority to U.S. Provisional Patent Application No. 63/444,104, filed on Feb. 8, 2023, the entire disclosure of which is incorporated herein by reference.

### BACKGROUND

Transposons, or jumping genes, were first discovered in maize by geneticist Barbara McClintock in the 1940s. Since then, scientists have shown that endogenous retroelements comprise 50% of the human genome and are oftentimes masked in clinical metagenomics samples due to highly repetitive sequences that are mostly inactive. Historically, human transposon signatures have been normalized in a metagenomics clinical sample to reduce sample analysis time. This may produce false negative results in cases of pathogen detection if in fact pathogenic genes are integrating in these regions and therefore masked.

Chimeras or chimeric sequences may include pathogenic transgenes integrated within the human genome. It is estimated that 15% of cancers are derived from viral pathogens with evidence of integration into the host genome, for example, for hepatitis B virus (HBV), human papillomavirus (HPV), Merkel cell polyomavirus (MCV), Epstein Barr virus (EBV) and human T-cell lymphotropic virus (HTLV). Current sequencing technologies such as the Basic Local Alignment Search Tool (BLAST), provided by the National Center for Biotechnology Information (NCBI), are largely manually driven processes that may not scale to the volume needed for probability determinations.

As an example, hepatocellular carcinoma (HCC) is the sixth most common cancer and third most frequent cause of cancer death worldwide, with chronic HBV and hepatitis C virus (HCV) infections being primary causes. Currently, patients identified as high risk for HCC are recommended to be monitored by ultrasound with or without alpha-fetoprotein (AFP) testing. Currently, there are no approved genomic biomarkers that can predict or diagnose HCC early enough to increase survival rates.

### SUMMARY

According to one aspect of the disclosure, a method for individualized metagenomic profiling comprises receiving, by a computing device, a genome sequence for an individual; mapping, by the computing device, the genome sequence to generate a genome map compared to a predetermined sample human genome; mapping, by the computing device, one or more chimeric sequences associated with an identified pathogen to the genome sequence to generate a chimera map; mapping, by the computing device, one or more active transposons to the genome sequence to generate a transposon map; and generating, by the computing device, a biomedical fingerprint associated with the individual by overlaying the genome map, the chimera map, and the transposon map.

In an embodiment, mapping the one or more chimeric sequences comprises performing a single pass query through the genome sequence. In an embodiment, the method further comprises mapping, by the computing device, an epigenetic profile to the genome sequence to generate an epigenetic map; wherein generating the biomedical fingerprint further comprises overlaying the genome map, the chimera map, the transposon map, and the epigenetic map. In an embodiment, mapping the epigenetic profile comprises mapping epigenetic markers comprising DNA methylation or chromatin structure. In an embodiment, mapping the genome sequence comprises identifying an insert, a deletion, or an inversion. In an embodiment, mapping the one or more chimeric sequences comprises identifying a chimeric sequence from a predetermined database of sequences indicative of human and pathogenic organisms. In an embodiment, mapping the one or more chimeric sequences comprises identifying at least one protein coding or non-coding region that includes two or more regions with non-overlapping species-level taxa predictions. In an embodiment, mapping the one or more transposons to the genome sequence comprises identifying active transposon coding.

In an embodiment, the method further comprises predicting, by the computing device, a disease diagnosis by inputting the biomedical fingerprint to a machine learning model of the computing device. In an embodiment, the machine learning model comprises a trained tree ensemble model. In an embodiment, the disease diagnosis comprises a disease severity prediction or a disease progression. In an embodiment, the method further comprises determining, by the computing device, a treatment regimen based on the disease diagnosis. In an embodiment, predicting the disease diagnosis comprises predicting the disease diagnosis based on a DNA methylation map and proximity of an active LINE-1 transposon to an oncogene associated with hepatocellular carcinoma (HCC).

According to another aspect, a computing device for individualized metagenomic profiling comprises a bioinformatics platform and a profile manager. The bioinformatics platform is to receive a genome sequence for an individual; map the genome sequence to generate a genome map compared to a predetermined sample human genome; map one or more chimeric sequences associated with an identified pathogen to the genome sequence to generate a chimera map; and map one or more active transposons to the genome sequence to generate a transposon map. The profile manager is to generate a biomedical fingerprint associated with the individual by an overlay of the genome map, the chimera map, and the transposon map.

In an embodiment, to map the one or more chimeric sequences comprises to perform a single pass query through the genome sequence. In an embodiment, the bioinformatics platform is further to map an epigenetic profile to the genome sequence to generate an epigenetic map; and to generate the biomedical fingerprint further comprises to overlay the genome map, the chimera map, the transposon map, and the epigenetic map. In an embodiment, to map the epigenetic profile comprises to map epigenetic markers that comprise DNA methylation or chromatin structure. In an embodiment, to map the genome sequence comprises to identify an insert, a deletion, or an inversion. In an embodiment, to map the one or more chimeric sequences comprises to identify a chimeric sequence from a predetermined database of sequences indicative of human and pathogenic organisms. In an embodiment, to map the one or more chimeric sequences comprises to identify at least one protein coding or non-coding region that includes two or more regions with non-overlapping species-level taxa predictions. In an embodiment, to map the one or more transposons to the genome sequence comprises to identify active transposon coding.

In an embodiment, the computing device further comprises a correlation manager to predict a disease diagnosis by input of the biomedical fingerprint to a machine learning model of the computing device. In an embodiment, the machine learning model comprises a trained tree ensemble model. In an embodiment, the disease diagnosis comprises a disease severity prediction or a disease progression. In an embodiment, the correlation manager is further to determine a treatment regimen based on the disease diagnosis. In an embodiment, to determine the disease diagnosis comprises to determine the disease diagnosis based on a DNA methylation map and proximity of an active LINE-1 transposon and a pathogenic insert to an oncogene associated with hepatocellular carcinoma (HCC).

According to another aspect, a method for diagnosing or obtaining a prognosis for a cancer comprises isolating or purifying DNA from a patient sample; bisulfite-treating the DNA; preparing a sequencing library from the bisulfite-treated DNA; hybridizing the DNA in the sequencing library with a probe panel to isolate a target gene from the DNA; amplifying the target gene; sequencing the target gene; and diagnosing or obtaining a prognosis for the cancer. In an embodiment, the cancer is caused by a Hepatitis B virus, a Hepatitis C virus, an Epstein Barr virus, or a human papilloma virus.

In an embodiment, the probe panel targets virus integration sites, LINE-1 integration sites, introns and exons of oncogenes or tumor suppressors, methylation, or a combination thereof. In an embodiment, the probe panel targets virus integration sites, LINE-1 integration sites, and methylation concurrently. In an embodiment, the patient sample is plasma. In an embodiment, the patient sample is blood. In an embodiment, the patient sample is liver tissue.

In an embodiment, the target gene is selected from the group consisting of TP53, TERT (including the promoter region), MLL4 (KMT2B), CCNE1, SENP5, ROCK1, FN1, ESPL1, SERCA1, ADAM12, PREX2, ANGPT1, ATM, ATR, CCNA2, SCFD2, DCC, OAZ2, ANO3, ENOX1, GRIK4, NPAT, SNCAIP, MYC, APOBEC3, SAMHD1, MOV10, HBx-LINE1, Rad21, genes whose rearrangements or mutation has been implicated directly in hepatocellular carcinoma, and combinations thereof. In an embodiment, the sequencing is Illumina NextSeq sequencing. In an embodiment, the sequencing is nanopore sequencing (MiniIon). In an embodiment, the sequencing is Single Molecule, Real-Time (SMRT) sequencing (PacBIO).

In an embodiment, the amplification is performed using the polymerase chain reaction. In an embodiment, the method is used to obtain a diagnosis. In an embodiment, the method is used to obtain a prognosis. In an embodiment, the DNA is circulating cell-free DNA (ccfDNA). In an embodiment, the DNA is genomic DNA.

In an embodiment, the method further comprises sequencing the DNA in the sequencing library to generate sequence data. Diagnosing or obtaining the prognosis for the cancer comprises identifying, by a computing device, HBV integration sites based on the sequence data; identifying, by the computing device, active LINE-1 integration sites based on the sequence data; identifying, by the computing device, hypomethylation sites based on the sequence data; determining, by the computing device, a mutation analysis for the oncogenebased on the sequence data; and determining, by the computing device, an HCC disease state progression based on the HBV integration sites, the active LINE-1 integration sites, the hypomethylation sites, and the mutation analysis.

In an embodiment, identifying the HBV integration sites comprises identifying a quantity and a location of the HBV integration sites; and identifying the active LINE-1 integration sites comprises identifying a quantity and a location of the active LINE-1 integration sites. In an embodiment, the hybridization probe panel further targets hepatitis C virus (HCV) and Epstein-Barr virus (EBV). In an embodiment, determining the HCC disease state progression comprises identifying HBV integration sites, active LINE-1 integration sites, and hypomethylation sites in proximity to the oncogene. In an embodiment, predicting the disease progression comprises classifying the disease progression as healthy, HBV-infected, HBV-associated cirrhosis, or HCC.

According to another aspect, a system for genomic analysis comprises a sequencer and a computing device. The sequencer is to sequence a prepared biological sample from an individual to generate sequence data, wherein the prepared biological sample is bisulfate treated and enriched with a hybridization probe panel that targets hepatitis B virus (HBV), LINE-1 transposon, and an oncogene associated with hepatocellular carcinoma (HCC). The computing device is to identify HBV integration sites based on the sequence data; identify active LINE-1 integration sites based on the sequence data; identify hypomethylation sites based on the sequence data; determine a mutation analysis for the oncogene based on the sequence data; and determine an HCC disease state progression based on the HBV integration sites, the active LINE-1 integration sites, the hypomethylation sites, and the mutation analysis.

In an embodiment, the prepared biological sample is a plasma sample comprising cell-free DNA. In an embodiment, the prepared biological sample is a liver tissue sample comprising genomic DNA. In an embodiment, the prepared biological sample is converted to a DNA sequencing library.

In an embodiment, to identify the HBV integration sites comprises identifying a quantity and a location of the HBV integration sites; and to identify the active LINE-1 integration sites comprises to identify a quantity and a location of the active LINE-1 integration sites. In an embodiment, the hybridization probe panel further targets hepatitis C virus (HCV) and Epstein-Barr virus (EBV). In an embodiment, the hybridization probe panel further targets a tumor suppressor gene associated with HCC. In an embodiment, to determine the HCC disease state progression comprises to identify HBV integration sites, active LINE-1 integration sites, and hypomethylation sites in proximity to the oncogene or the tumor suppressor gene. In an embodiment, to determine the disease progression comprises to classify the disease progression as healthy, HBV-infected, HBV-associated cirrhosis, or HCC.

### BRIEF DESCRIPTION OF THE DRAWINGS

The detailed description particularly refers to the accompanying figures in which:

FIG. 1 is a simplified block diagram of at least one embodiment of a system for individualized metagenomic profiling;

FIG. 2 is a simplified block diagram of at least one embodiment of an environment that may be established by a computing device of the system of FIG. 1;

FIG. 3 is a simplified flow diagram of at least one embodiment of a method for individualized metagenomic profiling that may be executed by the computing device of FIGS. 1 and 2;

FIG. 4 is a schematic diagram illustrating various metagenomic maps that may be generated by the method of FIG. 3;

FIG. 5 is a schematic diagram illustrating at least one embodiment of a biomedical fingerprint that may be generated based on the metagenomic maps of FIG. 4;

FIG. 6 is a schematic diagram illustrating additional metagenomic maps that may be generated by the method of FIG. 3;

FIG. 7 is a schematic diagram illustrating at least one embodiment of another biomedical fingerprint that may be generated based on the metagenomic maps of FIG. 6;

FIG. 8 is a schematic diagram illustrating at least one embodiment of individualized metagenomics profiling that may be performed by the system of FIGS. 1 and 2; and

FIG. 9 is a simplified flow diagram of at least one embodiment of a method for an individual metagenomics analysis technology that may be executed using the system of FIGS. 1 and 2.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but to the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C): (A and B); (B and C); or (A, B, and C). Similarly, items listed in the form of “at least one of A, B, or C” can mean (A); (B); (C): (A and B); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

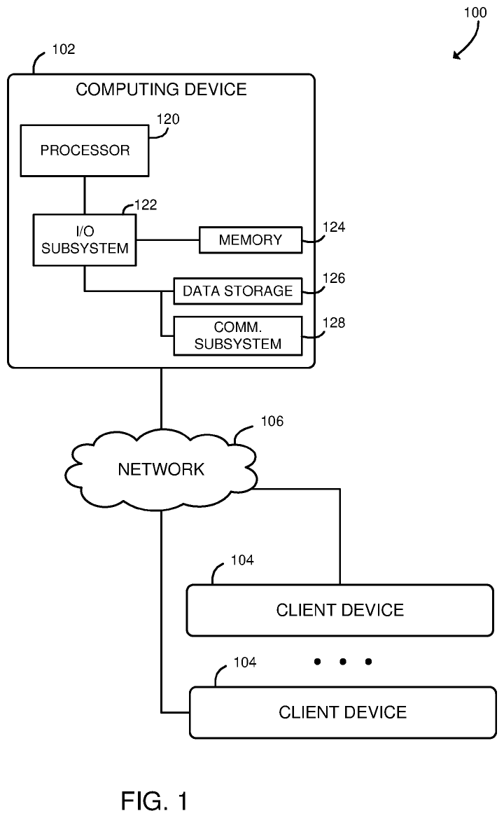

Referring now to FIG. 1, an illustrative system 100 includes a computing device 102 that may be in communication with one or more client devices 104 over a network 106. In use, as described further below, the computing device 102 receives a genome sequence for an individual (e.g., from a client device 104) and then screens the genome sequence in a single pass and generates a biomedical fingerprint associated with the individual. To perform this screening, the computing device 102 generates and integrates a genome map, a chimera map, a transposon map, and/or an epigenetic profile of the genome sequence. The computing device 102 may correlate the biomedical fingerprint with disease risk and/or progression and may determine an appropriate treatment regimen. Thus, the system 100 improves genetic sequencing and analysis by providing high-throughput metagenomic analysis incorporating chimera maps, transposon maps, epigenetic profiles, and/or other metagenomic information that is not analyzed by typical systems. For example, typical studies into risks associated with a pathogen's genetic integration into human chromosomes are manually driven with low throughput and study very specific instances of integration. Unlike typical studies, the system 100 provides high-throughput, reliable, and accurate chromosomal chimera detection for multiple pathogenic species, which is not possible using conventional manual techniques. Accordingly, the system 100 may reduce false negative and/or false positive results for pathogenic gene/organism detection, improve patient immunity and disease prediction, and otherwise may improve and inform research and care regimens for pathogens.

Additionally or alternatively, in some embodiments the system 100 may assess disease progression for hepatocellular carcinoma (HCC) with the use of a targeted hybridization panel to simultaneously detect HBV and LINE-1 transposon integration sites, oncogenes, tumor suppressors, and methylation status of ccfDNA and tissue specimens (e.g., liver tissue). Thus, the system 100 enables HCC early detection by assessment of HBV insertions, transposons, and aberrant methylome patterns concurrently, unlike typical tests that do not assess all three biomarkers concurrently. Further, unlike typical state-of-the art screening, the system 100 does not require imaging and thus reduces the cost and time needed for HCC diagnostic monitoring. Therefore, the system 100 provides improved early detection and reduced costs for testing. Additionally or alternatively, although described as focused on HCC prevention, the system 100 may be adapted to detect other virally driven cancers, such as cervical cancer caused by HPV, lymphomas associated with EBV, and other virally driven cancers.

Referring again to FIG. 1, the computing device 102 may be embodied as any type of device capable of performing the functions described herein. For example, the computing device 102 may be embodied as, without limitation, a server, a rack-mounted server, a blade server, a workstation, a network appliance, a web appliance, a desktop computer, a laptop computer, a tablet computer, a smartphone, a consumer electronic device, a distributed computing system, a multiprocessor system, and/or any other computing device capable of performing the functions described herein. Additionally, in some embodiments, the computing device 102 may be embodied as a “virtual server” formed from multiple computing devices distributed across the network 106 and operating in a public or private cloud. Accordingly, although the computing device 102 is illustrated in FIG. 1 as embodied as a single computing device, it should be appreciated that the computing device 102 may be embodied as multiple devices cooperating together to facilitate the functionality described below. As shown in FIG. 1, the illustrative computing device 102 includes a processor 120, an I/O subsystem 122, memory 124, a data storage device 126, and a communication subsystem 128. Of course, the computing device 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the computing device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the computing device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the computing device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the computing device 102 may be embodied as any communication circuit, device, or collection thereof, capable of enabling communications between the computing device 102 and other remote devices. The communication subsystem 128 may be configured to use any one or more communication technology (e.g., wireless or wired communications) and associated protocols (e.g., Ethernet, InfiniBand® Bluetooth®, Wi-Fi®, WiMAX, 3G LTE, 5G, etc.) to effect such communication.

The client device 104 is configured to access the computing device 102 and otherwise perform the functions described herein. The client device 104 may be embodied as any type of computation or computer device capable of performing the functions described herein, including, without limitation, a computer, a laptop computer, a notebook computer, a tablet computer, a mobile computing device, a wearable computing device, a multiprocessor system, a server, a rack-mounted server, a blade server, a network appliance, a web appliance, a distributed computing system, a processor-based system, and/or a consumer electronic device. Thus, the client device 104 includes components and devices commonly found in a computer or similar computing device, such as a processor, an I/O subsystem, a memory, a data storage device, and/or communication circuitry. Those individual components of the client device 104 may be similar to the corresponding components of the computing device 102, the description of which is applicable to the corresponding components of the client device 104 and is not repeated herein so as not to obscure the present disclosure.

Each of the computing device 102 and/or the client devices 104 may be configured to transmit and receive data with each other and/or other devices of the system 100 over the network 106. The network 106 may be embodied as any number of various wired and/or wireless networks. For example, the network 106 may be embodied as, or otherwise include, a wired or wireless local area network (LAN), a wired or wireless wide area network (WAN), a cellular network, and/or a publicly-accessible, global network such as the Internet. As such, the network 106 may include any number of additional devices, such as additional computers, routers, stations, and switches, to facilitate communications among the devices of the system 100.

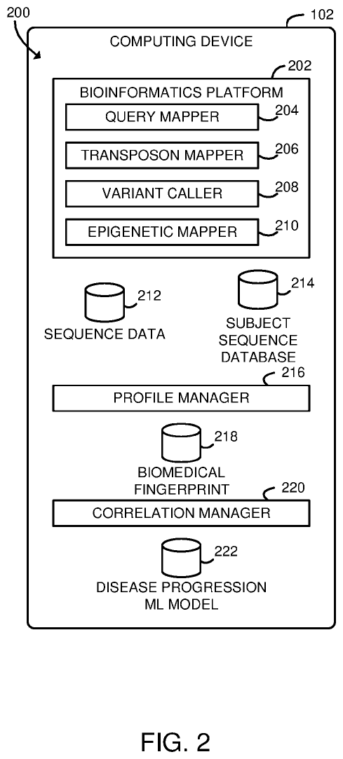

Referring now to FIG. 2, in the illustrative embodiment, the computing device 102 establishes an environment 200 during operation. The illustrative environment 200 includes a bioinformatics platform 202, a profile manager 216, and a correlation manager 220. The various components of the environment 200 may be embodied as hardware, firmware, software, or a combination thereof. As such, in some embodiments, one or more of the components of the environment 200 may be embodied as circuitry or a collection of electrical devices (e.g., bioinformatics platform circuitry 202, profile manager circuitry 216, and/or correlation manager circuitry 220). It should be appreciated that, in such embodiments, one or more of those components may form a portion of the processor 120, the memory 124, the data storage 126, and/or other components of the computing device 102.

The bioinformatics platform 202 is configured to receive a genome sequence for an individual. The genome sequence may be stored as or otherwise represented by sequence data 212, which may be embodied as nucleic acid sequence reads (and/or bisulfate treated sequence reads) from metagenomics Next Generation Sequence (mNGS) data (e.g., FASTQ files) from sequences produced by Illumina, Nanopore (MinION), Single Molecule, Real-Time (SMRT) sequencing (PacBio), Ion Torrent (Thermo-Fisher) sequencers, including older generation sequencers. The bioinformatics platform 202 is further configured to map the genome sequence to generate a genome map compared to a predetermined sample human genome, to map one or more chimeric sequences associated with an identified pathogen to the genome sequence to generate a chimera map, and to map one or more active transposons to the genome sequence to generate a transposon map. Sequencing the genome sequence may include identifying an insert, a deletion, or an inversion. Mapping the one or more chimeric sequences may include identifying a chimeric sequence from a predetermined subject sequence database 214 of sequences indicative of pathogenic organisms. For example, mapping the one or more chimeric sequences may include identifying at least one protein coding or non-coding region that includes two or more regions with non-overlapping species-level taxa predictions. Mapping the one or more chimeric sequences may be performed with a single pass query through the genome sequence. Mapping the one or more transposons to the genome sequence may include identifying active transposon coding. In some embodiments, the bioinformatics platform 202 may be further configured to map an epigenetic profile to the genome sequence to generate an epigenetic map. Mapping the epigenetic profile may include mapping epigenetic markers such as DNA methylation or chromatin structure. In some embodiments, one or more of those functions of the bioinformatics platform 202 may be performed by one or more sub-components, applications, or tools, such as a query mapper 204, a transposon mapper 206, a variant caller 208, and/or an epigenetic mapper 210.

The profile manager 216 is configured to generate a biomedical fingerprint 218 associated with the individual by overlaying the genome map, the chimera map, and the transposon map. In some embodiments, generating the biomedical fingerprint 218 may include overlaying the genome map, the chimera map, the transposon map, and the epigenetic map.

The correlation manager 220 is configured to predict a disease diagnosis by inputting the biomedical fingerprint 218 into a machine learning model 222, which in some embodiments may be a trained tree ensemble model. The disease diagnosis may include a disease severity prediction or a disease progression. For example, in an embodiment predicting the disease diagnosis may include predicting the disease diagnosis based on a DNA methylation map and proximity of a pathogenic insert (e.g., HBV chimera) and/or an active LINE-1 transposon to an oncogene associated with HCC. In some embodiments, the correlation manager 220 is further configured to determine a treatment regimen based on the disease diagnosis.

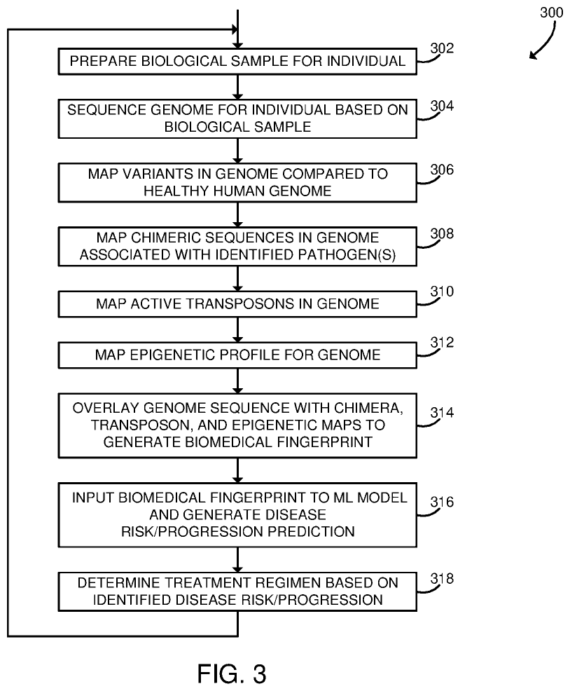

Referring now to FIG. 3, in use, the computing device 102 may execute a method 300 for individualized metagenomic profiling. It should be appreciated that, in some embodiments, the operations of the method 300 may be performed by one or more components of the environment 200 of the computing device 102 as shown in FIG. 2. The method 300 begins with block 302, in which a biological sample for an individual is prepared for genome sequencing. For example, in some embodiments ccfDNA may be extracted from a plasma, or genomic DNA may be extracted from a tissue sample from the individual. The plasma sample may be a minimally invasive technique for analysis and monitoring. In some embodiments, the biological sample may be prepared for epigenetic profiling, for example by performing bisulfate conversion, in which unmethylated cytosines in the sample are changed to uracil, which are read as thymine when sequenced. Thus, by comparison to sequences that have not undergone bisulfate conversion, a methylome or other map of DNA methylation may be determined.

In block 304, the biological sample is sequenced, which generates genome sequence data 212, which is received by the computing device 102. The sequence data 212 includes nucleotide sequence data for an individual (e.g., a patient), and may be generated after sequencing the nucleic acids by using any suitable sequencing method including Next Generation Sequencing (e.g., using Illumina, ThermoFisher, PacBio or Oxford Nanopore Technologies sequencing platforms), sequencing by synthesis, pyrosequencing, nanopore sequencing, or modifications or combinations thereof can be used. Methods for sequencing nucleic acids are also well-known in the art and are described in Sambrook et al., “Molecular Cloning: A Laboratory Manual”, Cold Spring Harbor Laboratory Press, incorporated herein by reference. Accordingly, the sequence data 212 may include nucleic acid sequence reads from metagenomics Next Generation Sequence (mNGS) data (e.g., FASTQ files) from sequences produced by Illumina, Nanopore (MinION), Single Molecule, Real-Time (SMRT) sequencing (PacBio), Ion Torrent (Thermo-Fisher) sequencers, including older generation sequencers. In an embodiment, the query sequence data may be received from one or more client devices 104, for example through submission to a web application or other server application executed by the computing device 102. Additionally, or alternatively, in some embodiments the computing device 102 may receive the query sequence data from a local or remote user through a user interface, from a sequencing machine, or from another sequence source.

In block 306, the computing device 102 maps variants of the sequence data 212 compared to healthy human genome samples. For example, the computing device 102 may align, map, or otherwise identify locations for insertions, deletions, inversions, or other variants compared to a known human genome sequence. For example, in an embodiment, the genome may be mapped against a reference genome such as the HG38 human reference genome. In some embodiments, the computing device may map the sequence data compared to multiple known human genome sequences.

In block 308, the computing device 102 aligns, maps, or otherwise identifies the location of chimeric sequences including sequences from one or more identified pathogens. Each chimeric sequence includes genetic material originating from a virus or other non-human organism (e.g., a human-pathogen insert). To perform this alignment, the computing device 102 may determine an alignment of one or more query sequences from the sequence data 212 against the subject sequence database 214. The database 214 may include a predetermined sequence of concern database that includes genetic sequence data for certain known pathogens. Additionally or alternatively, the subject sequence database 214 may include one or more customizable indexes used for sequence alignment including, for example, human genome and pathogen genomes from an NCBI nucleotide database or other predetermined subject sequence database or index. Determining the alignment identifies sequences within the subject sequence database 214 that are similar to the query sequence. Additionally, when determining the alignment, the computing device 102 may determine a score, a similarly value, a confidence value, or another quantitative measure of similarity between the query sequence and one or more sequences within the subject sequence database 214. The computing device 102 may use any local alignment, global alignment, or other genetic sequence alignment algorithm to align the query sequences against the subject sequence database 214.

As described above, chimeric sequences may be determined by matching against the subject sequence database 214, which includes genetic sequence data for certain known pathogens, such as SARS-COV-2, Human herpesvirus 6 (HHV6), Human gamma herpesvirus 4 (Epstein Barr), Pseudomonas aeruginosa, Leptospira interrogans, Borna disease virus (BDV), Herpes simplex virus type 1 (HSV-1), Varicella zoster virus (VZV), Cytomegalovirus (CMV), Human immunodeficiency virus (HIV), Filovirus (EBOLA, Marburg), or other human-pathogen integration events. Chimeras may occur when sequences from two different species are present in a single sequence read. Each identified chimeric sequence may include at least one protein coding or non-coding region and more than 2 regions with non-overlapping species level taxa identifier predictions. Such pathogen integration events may occur from mechanisms associated with reverse-transcription (e.g., RNA viruses) or transposon activities from human genes or pathogen genes. In some embodiments, chimeric sequences may be identified using the UltraSEQ universal bioinformatics platform developed by Battelle Memorial Institute, which can rapidly target pathogenic sequences that are chimeras within the human genome and provide gene function or other pathogenic properties of the aligned sequences using the subject sequence database 214. As an example, sequences of interest may include SARS-COV-2 sequences such as the Nucleocapsid protein (NC) located at the 3′ end of the SARS-COV-2 genome. UltraSEQ has tunable parameters to quickly separate higher confidence results from lower confidence results (e.g., top alignment score, threat confidence, or other parameters).

In block 310, the computing device 102 maps active transposons in the sequence data 212. Transposons or transposable elements (TEs) are sequences of DNA that can change their position within the genome. Such transposons form a large proportion of the human genome. For example, the LINE-1 transposon may form about 20% of the human genome. However, a large proportion of those transposons are truncated and/or inactive. For example, in some cases, less than 200 out of about 500,000 instances of the LINE-1 may be active. In order to decipher and map locations of active transposons (e.g., LINE-1), the subject sequence database 214 may include for example, LINE-1 endonuclease recognition sequences, LINE-1 target-site duplications, and human LINE-1 proteins (ORF1p, ORF2p). Human TE LINE-1 encodes two proteins: ORF1p, an RNA binding protein, and ORF2p, a replicase (endonuclease and reverse transcriptase). If both of those proteins are present in non-truncated form, this may be an indicator of an active TE.

In some embodiments, in block 312 the computing device 102 may map an epigenetic profile of the sequence data 212. The epigenetic profile includes maps, data, or other non-sequence information that may affect expression, regulation, or other factors of the sequence data. For example, in some embodiments, the epigenetic map may include a mapping of methylated and/or unmethylated cytosines or other methylome data.

In block 314, the computing device 102 overlays the genome map with the chimera map, the transposon map, and the epigenetic map (if available) to generate a biomedical fingerprint 218 associated with the individual. The biomedical fingerprint 218 allows for concurrent analysis of each stream of information, including genetic sequence, chimera, transposon, and epigenetic profiles. Accordingly, the biomedical fingerprint 218 may include features indicative of the relative locations of HBV insertions and LINE-1 transposons to regions of hypomethylation, oncogenes, and tumor suppressors; the diversity of the inserted sequences; proximity to and/or addition of promoter sequences relative to HB V and LINE-1 sites; frequency of hypomethylation, especially with respect to oncogenes and tumor suppressors, and other features.

In block 316, the computing device 102 correlates disease risks or progression based on the biomedical fingerprint. The computing device 102 may input the biomedical fingerprint 218 to the machine learning model 222 in order to generate a predicted disease risk and/or a predicted disease progression. The machine learning model 222 may be trained based on one or more statistical correlations based on known data in order to predict risk for one or more particular diseases based on the combined chimera map, transposon map, and/or epigenetic map. The computing device 102 may determine correlated disease risks and/or immunity for one or more infectious diseases, chronic illnesses, autoimmune diseases, cancer, or other diseases. As an example, the computing device 102 may predict disease state related to HCC as being one of healthy, HBV infected, cirrhosis, or HCC. In other embodiments the computing device 102 may predict disease state for endometriosis, Lyme disease, Long-Covid, Chronic fatigue syndrome, Fibromyalgia, or other chronic illnesses or autoimmune diseases.

The machine learning model 222 may be trained using sample data, for example data from an ancestrally diverse cohort consisting of 160 plasma samples (40 healthy, 40 HBV infected, 40 HBV-associated cirrhosis, and 40 HBV-associated HCC). Visual examination (e.g., scatterplot matrices) and quantitative analysis (e.g., clustering algorithms) indicate that the features of the biomedical fingerprint 218 show separation between the four phenotypes represented across the cohort (healthy, HBV, cirrhosis, HCC) and are not strongly associated with factors relating to ancestry, age, and other demographics. The machine learning model 222 is trained for prediction or classification of disease state (e.g., healthy, HBV-infected, HBV-associated cirrhosis, or HCC) using the biomedical fingerprint 218 described above as input features for the predictors. In some embodiments, the machine learning model 222 may be a tree-based ensemble model (e.g., random forests) or a gradient boosting model, which are robust to monotone transformations of features and may be successful in a variety of scenarios. In some embodiments, the machine learning model 222 may be regularized regression model such as a Sparse-Group LASSO, which may allow for selection of a subset of features and data sources (e.g., HBV chimera, LINE-1 transposon, and/or methylation).

In block 318, the computing device 102 may determine a treatment regimen based on the identified disease risk and/or progression. For example, the computing device 102 may recommend a predetermined treatment regimen based on the identified disease risk or progression. After determining the treatment regimen, the method 300 loops back to block 302, in which the computing device 102 may continue sequencing genetic data and generating biomedical fingerprints. For example, the computing device 102 may generate biomedical fingerprints for additional individuals. Additionally or alternatively, the computing device 102 may generate additional biomedical fingerprints for the same individual over time, allowing changes in the chimera map, transposon map, and/or epigenetic map to be monitored over time.

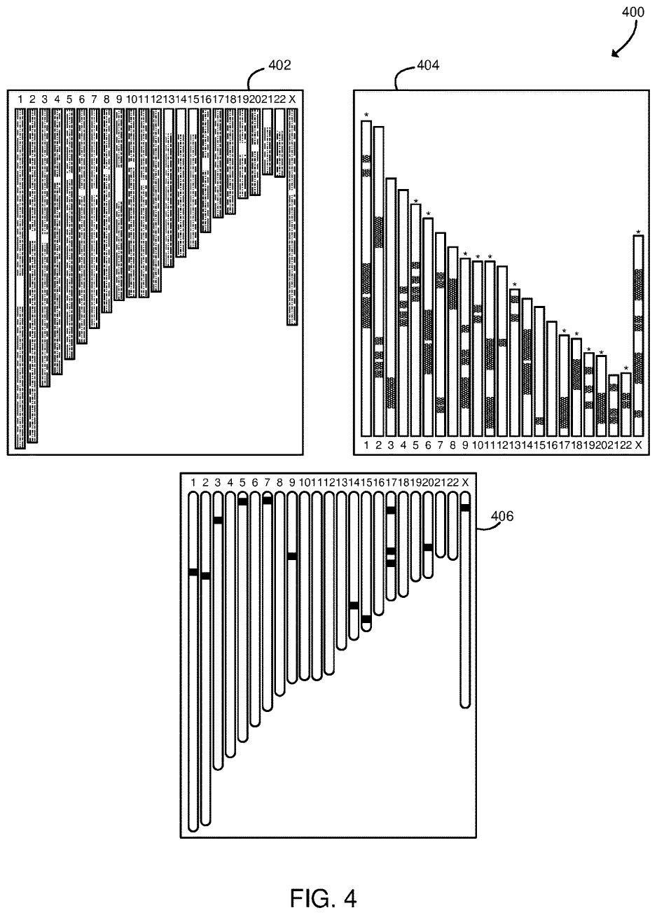

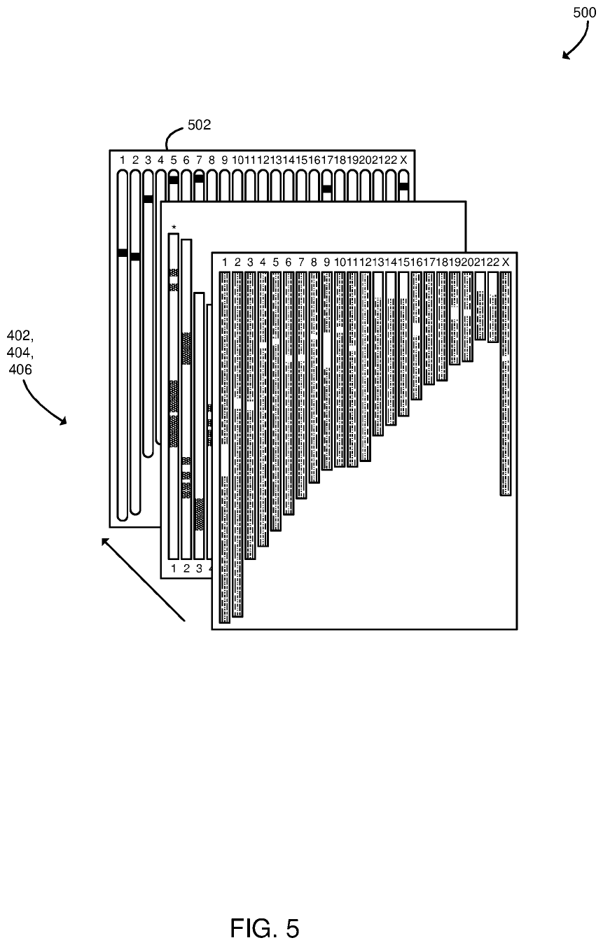

Referring now to FIG. 4, schematic diagram 400 illustrates at least one potential embodiment of metagenomics maps that may be generated by the system 100. Illustratively, the diagram 400 shows a genome map 402, a chimera map 404, and a transposon map 406. The illustrative genome map 402 illustrates variants within healthy human genome with inserts, deletions, and inversions identified. The chimera map 404 illustrates Human Herpesvirus 6 (HHV6) integration events (or other pathogens) within the genome. The transposon map 406 identifies the location of transposons identified in ovarian cancer cells (or other types of cancer). Referring now to FIG. 5, schematic diagram 500 illustrates at least one embodiment of a biomedical fingerprint based on the metagenomics maps of FIG. 4. Illustratively, the diagram 500 shows a biomedical fingerprint 502 that integrates the genome map 402, the chimera map 404, and the transposon map 406.

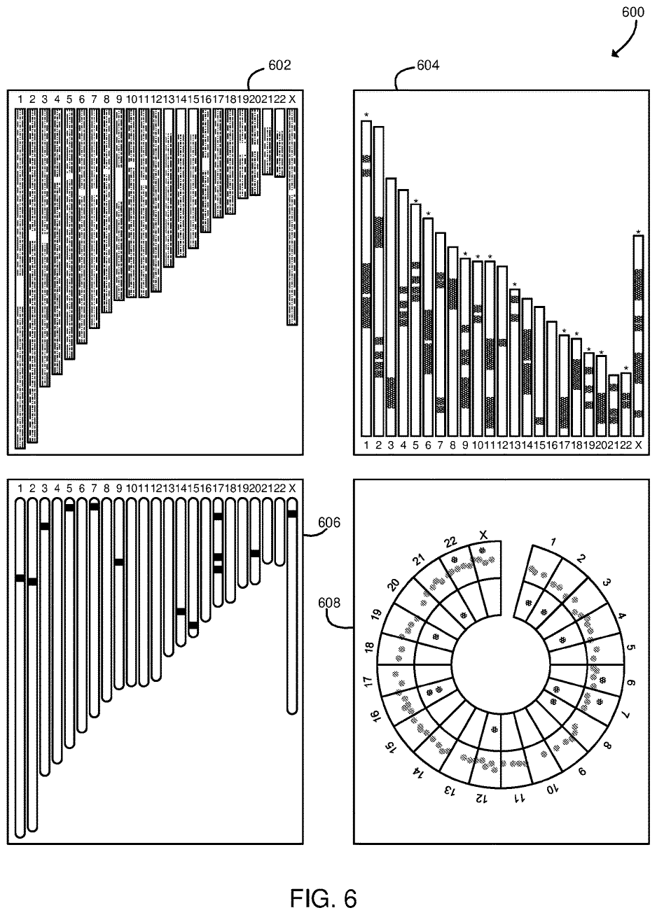

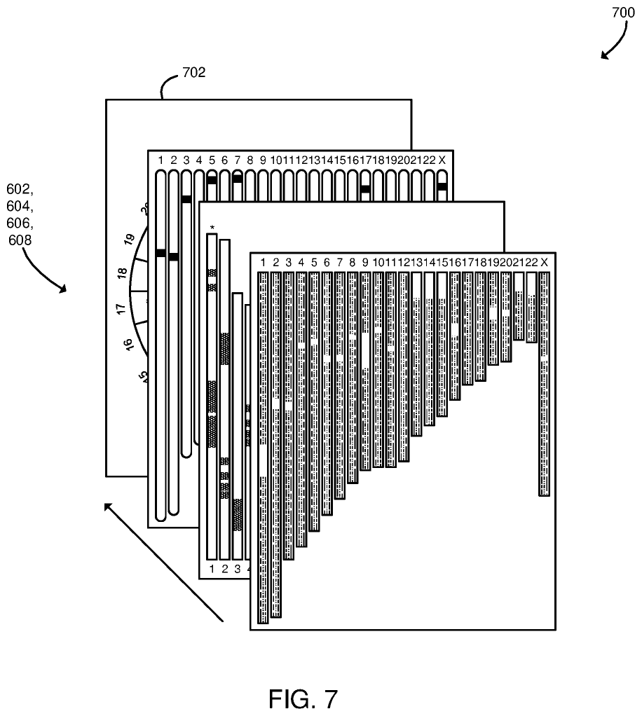

Referring now to FIG. 6, schematic diagram 600 illustrates at least one potential embodiment of metagenomics maps that may be generated by the system 100. Illustratively, the diagram 600 shows a genome map 602, a chimera map 604, a transposon map 606, and an epigenetics map 608. The genome map 602, the chimera map 604, and the transposon maps 606 are similar to the maps 402, 404, 406 shown in FIG. 4 and described above. The epigenetic map 608 shows methylation sites for the genome, including locations of hypomethylation. Referring now to FIG. 7, schematic diagram 700 illustrates at least one embodiment of a biomedical fingerprint based on the metagenomics maps of FIG. 6. Illustratively, the diagram 700 shows a biomedical fingerprint 702 that includes the genome map 602, the chimera map 604, the transposon map 606, and the epigenetic map 608.

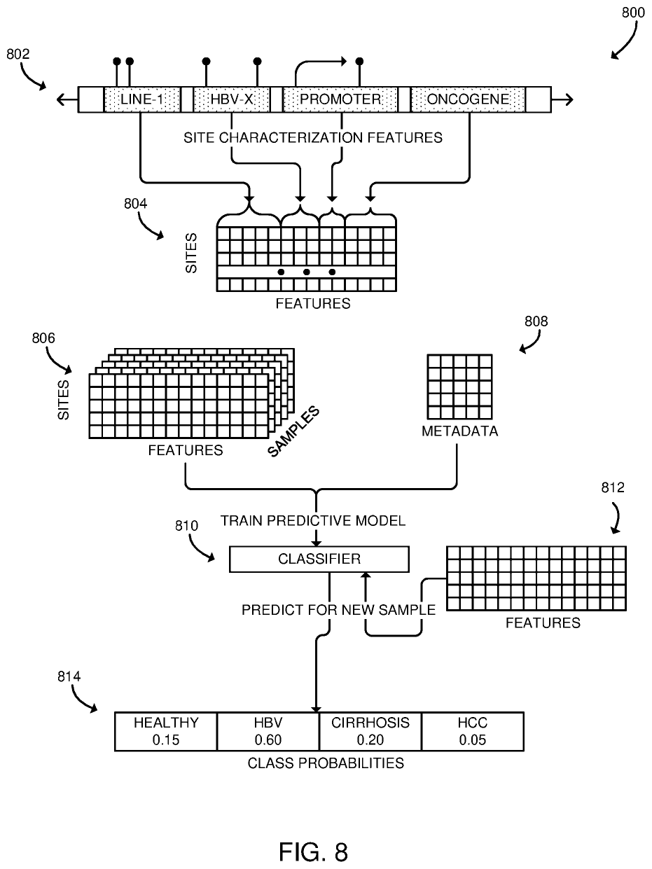

Referring now to FIG. 8, diagram 800 illustrates one potential embodiment of metagenomics profiling and prediction that may be performed by the system 100. The diagram 800 illustrates an insertion site 802. A biomedical fingerprint may be generated by generating one or more site characterization features of the insertion site 802. Such site characterization features may include the presence of a LINE-1 insertion, a LINE-1 methylation extent, a HBV sub-genotype, an HBV methylation extent, proximity of a promoter to a known oncogene, a promoter methylation extent, and other characterization features. The characterization features for multiple insertion sites from a sample may be stored in a sample feature table 804, which illustratively organizes sites into rows and site characterization features into columns.

In order to perform modeling and prediction, samples feature tables may be generated for multiple samples and stored into training data 806. The training data 806 and training sample metadata 808 (e.g., ethnicity, gender, or other data relating to sampled individuals) may be used to train a predictive model, which is illustratively a classifier 810. The classifier 810 may be a tree ensemble model such as the machine learning model 222 described above. After training, the classifier 810 may use new sample feature data 812 as input to generate a prediction 814. The prediction 814 is illustratively class probabilities for the disease progression classes (i.e., healthy, HBV infected, cirrhosis, and HCC).

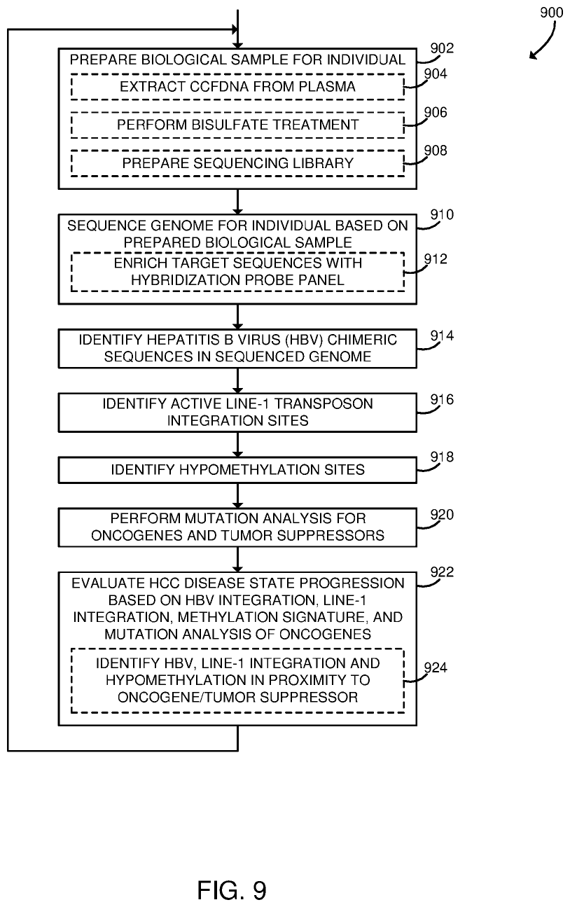

Referring now to FIG. 9, in use, the computing device 102 may execute a method 900 for an individual metagenomic analysis technology. It should be appreciated that, in some embodiments, the operations of the method 900 may be performed by one or more components of the environment 200 of the computing device 102 as shown in FIG. 2. In various embodiments, a patient sample can be tested as described herein. The patient sample can comprise human body fluids including, but not limited to, plasma, urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, lymph fluid, and whole blood, or serum, or any other suitable human patient sample (e.g., tissue). In one aspect, ccfDNA can be isolated and is the patient sample for analysis.

In one illustrative aspect, the nucleic acids (e.g., ccfDNA) in the patient sample are extracted and purified for analysis. In various embodiments, the preparation of the nucleic acids (e.g., DNA or RNA) can involve rupturing the cells that contain the nucleic acids and isolating and purifying the nucleic acids (e.g., DNA or RNA) from the lysate, or can involve isolating circulating cell-free DNA. Techniques for rupturing cells and for isolation and purification of nucleic acids (e.g., DNA or RNA) are well-known in the art. In one embodiment, for example, nucleic acids may be isolated and purified by rupturing cells using a detergent or a solvent, such as phenol-chloroform. In another aspect, nucleic acids (e.g., DNA, such as ccfDNA or RNA) may be separated from the lysate by physical methods including, but not limited to, centrifugation, pressure techniques, or by using a substance with an affinity for nucleic acids (e.g., DNA or RNA), such as, for example, beads that bind nucleic acids. In one embodiment, after sufficient washing, the isolated, purified nucleic acids may be suspended in either water or a buffer. In one embodiment, “isolated” means that the nucleic acids are removed from their normal environment (e.g., a nucleic acid is removed from the genome of an organism). In another aspect, “purified” means the nucleic acids are substantially free of other cellular material, or culture medium, or other chemicals used in the extraction process. In other embodiments, commercial kits are available, such as Qiagen™, Nuclisensm™, and Wizard™ (Promega), and Promegam™ for extraction, isolation, and purification of nucleic acids. Methods for preparing nucleic acids and for purifying and sequencing nucleic acids are also described in Green and Sambrook, “Molecular Cloning: A Laboratory Manual”, 4th Edition, Cold Spring Harbor Laboratory Press, (2012), incorporated herein by reference.

In one illustrative aspect, a sequencing library can be prepared, and the nucleic acids can be sequenced using any suitable sequencing method. In one embodiment, the target sequencing library can be prepared from bisulfite-treated ccfDNA. In one aspect, libraries can be pooled and concentrated before sequencing. Methods for library preparation and for sequencing are described in Green and Sambrook, “Molecular Cloning: A Laboratory Manual”, 4th Edition, Cold Spring Harbor Laboratory Press, (2012), incorporated herein by reference.

In one embodiment, probes, such as a probe panel, can be used to isolate target genes before sequencing. The probe panel can target, for example, virus integration sites, LINE-1 integration sites, introns and exons of oncogenes or tumor suppressors, methylation, or a combination thereof. In one aspect, the probe panel targets virus integration sites, LINE-1 integration sites, and methylation concurrently. In one embodiment, the probes can be used in a hybridization method, such as exome/targeted hybridization sequencing. In one aspect, hybridization can be performed using streptavidin sequence probes, for example, to bind the nucleic acids of interest, e.g., the target genes. In this illustrative embodiment, other sequences are removed from the library, and the target genes are amplified prior to sequencing, for example using the polymerase chain reaction.

Probes, or a probe panel, can be made by methods well-known in the art, including synthesis and recombinant methods. Such techniques are described in Sambrook et al., “Molecular Cloning: A Laboratory Manual”, 3rd Edition, Cold Spring Harbor Laboratory Press, (2001), incorporated herein by reference. Probes in the probe panel described herein can also be made commercially (e.g., Blue Heron, Bothell, WA 98021). Techniques for purifying or isolating probes, primers for amplification, or the nucleic acids for analysis described herein are well-known in the art. Such techniques are also described in Sambrook et al., “Molecular Cloning: A Laboratory Manual”, 3rd Edition, Cold Spring Harbor Laboratory Press, (2001), incorporated herein by reference.

In one aspect, the target genes can be in ccfDNA. In one aspect, the target gene can be selected from, but not limited to, the group consisting of TP53, TERT (including the promoter region), MLL4 (KMT2B), CCNE1, SENP5, ROCK1, FN1, ESPL1, SERCA1, ADAM12, PREX2, ANGPT1, ATM, ATR, CCNA2, SCFD2, DCC, OAZ2, ANO3, ENOX1, GRIK4, NPAT, SNCAIP, MYC, APOBEC3, SAMHD1, MOV10, HBx-LINE1, Rad21, genes whose rearrangements or mutation has been implicated directly in HCC, and combinations thereof.

In one embodiment, the target gene(s) can be sequenced and a diagnosis or a prognosis for a cancer can then be determined. Sequencing can be done by Next Generation Sequencing, sequencing by synthesis, pyrosequencing, nanopore sequencing, or modifications or combinations thereof, for example. In various aspects, the cancer can be caused by a HBV, a HCV, an EBV, or a HPV. In one embodiment, the method described herein enables a diagnostic accuracy and sensitivity higher than the current AFP biomarker assay (63% sensitivity Tzartzeva 2018a, Tzartzeva 2018b, Chen 2020, Cerrito 2022) for early detection.

The method 900 begins with block 902, in which a biological sample for an individual is prepared for genome sequencing. In some embodiments, in block 904 ccfDNA may be extracted from a plasma sample from the individual. This may be a minimally invasive collection for analysis and monitoring. In other embodiments, the biological sample may be a tissue sample. For example, in some embodiments genomic DNA may be extracted from a liver tissue sample from the individual. In some embodiments, in block 906 the biological sample is prepared for epigenetic profiling by performing bisulfate conversion, in which unmethylated cytosines in the sample are changed to uracil, which are read as thymine when sequenced. Thus, by comparison to sequences that have not undergone bisulfate conversion, a methylome or other map of DNA methylation may be determined. It should be understood that in some embodiments bisulfate conversion may not be necessary for certain sequence data (e.g., PacBio or Nanopore sequence data). In some embodiments, in block 908 the biological sampling may be converted into a sequencing library. For example, the ccfDNA sample may be fragmented into shorter segments of DNA, and specialized adapters may be added to both ends of each DNA fragment. The particular format or other techniques required to generate the sequencing library may depend on the particular DNA sequencer in use.

In block 910, the genome for the individual is sequenced using the prepared biological sample. Sequencing generates nucleotide sequence data 212 for an individual (e.g., a patient), and may include nucleic acid sequence reads from metagenomics Next Generation Sequence (mNGS) data (e.g., FASTQ files) from sequences produced by Illumina, Nanopore (MinION), Single Molecule, Real-Time (SMRT) sequencing (PacBio), Ion Torrent (Thermo-Fisher) sequencers, including older generation sequencers. To further prepare the sequence data 212, paired end read clusters may first be cleaned by removing adapters and performing quality and length filtering, for example using Trimmomatic. The average insert size across all clusters may be estimated, for example using Picard Tools. In some embodiments, in block 912, multiple predetermined target sequences may be captured, amplified, and enriched in the biological sample with a hybridization probe panel. The hybridization probe panel may target and tile across the full genomes of the most common virus genotypes (e.g., HBV, HCV, and EBV), LINE-1, and the introns and exons of genes that play a role in oncogenesis, including oncogenes and tumor suppressors which have been associated with HCC. The panel of human genes selected includes genes that have been identified as HBV integration hotspots in HCC or cirrhosis (including but not limited to TP53, TERT (including the promoter region), MLL4 (KMT2B), CCNE1, SENP5, ROCK1, FN1, ESPL1, SERCA1, ADAM12, PREX2, ANGPT1, ATM, ATR, CCNA2, SCFD2, DCC, OAZ2, ANO3, ENOX1, GRIK4, NPAT, SNCAIP, MYC, APOBEC3, SAMHD1, MOV10, HBx-LINE-1, and Rad21) and genes whose rearrangements or mutation has been implicated directly in HCC, which may cover about 100 (or more) human genes. In other embodiments the probe panel may target a different number of genes (e.g., about 5 genes, 100 genes, 105 genes, 600 genes, or a different number) based on cost, complexity, or other factors. The probe panel is compatible with bisulfite treated DNA, enabling the ability to monitor methylation changes along with insertions and mutations at the target sites. Additionally or alternatively, although hybridization capture is illustrated in FIG. 9 as being performed as a part of sequencing (e.g., hybrid-capture sequencing or target-enrichment sequencing), it should be understood that in some embodiments hybridization capture and enrichment may be performed as part of the sample preparation described in connection with block 902 or at other times. In some embodiments, hybridization capture may be an optional step that is not performed in all cases.

In block 914, the computing device 102 identifies HBV chimeric sequences in the sequenced genome using the sequence data 212. It has been shown that HBV integration into the human genome randomly occurs during infection, cirrhosis, and HCC, with more than 8,800 unique HBV integration sites identified, and clonal insertions developing in HCC when HBV integrates in oncogenes or causes recombination events that increases expression of oncogenes. To identify chimeric sequences, the computing device 102 may search the sequence data 212 for sequences that contain at least one protein coding or non-coding region and more than two regions with non-overlapping species level taxa identifier predictions (e.g., human and viral fragment). In some embodiments, HBV chimeric sequences may be identified using an UltraSEQ bioinformatic platform, developed by Battelle Memorial Institute. In use, UltraSEQ aligns reads to a set of reference databases including the UniRef100 and a user-configurable set of genomes. Utilizing an innovative, information-theory based taxonomy classification algorithm, UltraSEQ has been demonstrated to accurately classify metagenomics samples from a variety of sources, including over 407 clinical samples across 10 independent diagnostics studies with an accuracy of 91%.

In block 916, the computing device 102 identifies active LINE-1 transposon integration sites in the sequence data 212. LINE-1 activity has also been associated with HCC through disruption of tumor suppressors or activation of oncogenes, with evidence of about 329 full-length and potentially active instances of LINE-1 (out of more than 500,000 copies). Current human genomic analysis bioinformatic software typically discards sequences derived from transposable elements, which represent up to 50% of the human genome and pathogenic sequences. In contrast, the computing device 102 screens the entire genome for LINE-1 transposons. Next, read clusters may be rapidly downselected for those containing candidate viral or LINE-1 inserts by aligning against the human reference genome (hg38) and a database of viral genomes and LINE-1. Clusters returning alignments to both human and a viral genome or LINE-1 databases will be retained since they contain viral or LINE-1 integration into the human genome. The result of this pipeline will be the quantity and the location of HBV and LINE-1 insert events.

In block 918, the computing device 102 identifies hypomethylation sites in the sequence data 212. It has been shown that HB V infection and integration causes hypomethylation to occur in the human genome, which can enable LINE-1 activation. The computing device 102 may, for example, use one or more bioinformatics tools to map bisulfite reads and calling methylation and/or identify differentially methylated regions in the sequence data 212.

In block 920, the computing device 102 performs mutation analysis for oncogenes and tumor suppressors in the sequence data 212. For example, the computing device 102 may identify particular mutations (e.g., insertions, deletions, base changes, or other mutations) associated with introns and exons of genes that play a role in oncogenesis, including oncogenes and tumor suppressors which have been associated with HCC. The panel of human genes selected may include genes that have been identified as HBV integration hotspots in HCC or cirrhosis (including but not limited to TP53, TERT (including the promoter region), MLL4 (KMT2B), CCNE1, SENP5, ROCK1, FN1, ESPL1, SERCA1, ADAM12, PREX2, ANGPT1, ATM, ATR, CCNA2, SCFD2, DCC, OAZ2, ANO3, ENOX1, GRIK4, NPAT, SNCAIP, MYC, APOBEC3, SAMHD1, MOV10, HBx-LINE-1, and Rad21) and genes whose rearrangements or mutation has been implicated directly in HCC, covering about 100 or more human genes.

In block 922, the computing device 102 evaluates HCC disease state progression based on the HBV integrations, the LINE-1 integrations, the hypomethylation signature, and the mutation analysis described above. For example, the computing device 102 may determine HCC disease state progression based on statistically significant relationships to features present in the combined HBV integration, LINE-1 integration, hypomethylation signature, and mutation analysis data. In some embodiments, in block 924, the computing device 102 may identify HBV and LINE-1 integration sites and hypomethylation sites that are in proximity to one or more known oncogenes or tumor suppressor genes. The presence of those features may indicate progression of HCC. Statistically significant relationships may be identified using sample data from an ancestrally diverse cohort consisting of 160 plasma samples (40 healthy, 40 HBV infected, 40 HBV-associated cirrhosis, and 40 HBV-associated HCC) and compared to 20 liver tissue samples (5 each of healthy, HBV infected, HBV-associated cirrhosis and HBV-associated HCC). Each data track (methylation, HBV integrations, LINE-1 integrations, and mutations) may be analyzed individually to visualize and qualitatively assess the stronger signals that differentiate the clinical cohorts (healthy, HBV infected, cirrhosis, and HCC), followed by ANOVA or Chi-squared tests (since the sample size is large). The P-values from those statistical tests may be used to identify markers with statistically significant abundances between the cohort phenotypes (controlling for the family-wise false discovery rate). The P-values from those statistical tests may also serve as heuristics to rank individual markers. After evaluating the disease state progression, the method 900 loops back to block 902, in which the computing device 102 may continue performing metagenomics analysis. For example, the computing device 102 may perform analysis for additional individuals. Additionally or alternatively, the computing device 102 may perform analysis for the same individual over time, allowing for HCC monitoring and/or screening over time.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A method for individualized metagenomic profiling, the method comprising:
receiving, by a computing device, a genome sequence for an individual;
mapping, by the computing device, the genome sequence to generate a genome map compared to a predetermined sample human genome;
mapping, by the computing device, one or more chimeric sequences associated with an identified pathogen to the genome sequence to generate a chimera map;
mapping, by the computing device, one or more active transposons to the genome sequence to generate a transposon map; and
generating, by the computing device, a biomedical fingerprint associated with the individual by overlaying the genome map, the chimera map, and the transposon map.

2. The method of claim 1, wherein mapping the one or more chimeric sequences comprises performing a single pass query through the genome sequence.

3. The method of claim 1, wherein:
the method further comprises mapping, by the computing device, an epigenetic profile to the genome sequence to generate an epigenetic map; and
generating the biomedical fingerprint comprises overlaying the genome map, the chimera map, the transposon map, and the epigenetic map.

4. The method of claim 3, wherein mapping the epigenetic profile comprises mapping epigenetic markers comprising DNA methylation or chromatin structure.

5. The method of claim 1, wherein mapping the genome sequence comprises identifying an insert, a deletion, or an inversion.

6. The method of claim 1, wherein mapping the one or more chimeric sequences comprises identifying a chimeric sequence from a predetermined database of sequences indicative of human and pathogenic organisms.

7. The method of claim 1, wherein mapping the one or more chimeric sequences comprises identifying at least one protein coding or non-coding region that includes two or more regions with non-overlapping species-level taxa predictions.

8. The method of claim 1, wherein mapping the one or more transposons to the genome sequence comprises identifying active transposon coding.

9. The method of claim 1, further comprising predicting, by the computing device, a disease diagnosis by inputting the biomedical fingerprint to a machine learning model of the computing device.

10. The method of claim 9, wherein the machine learning model comprises a trained tree ensemble model.

11. The method of claim 9, wherein the disease diagnosis comprises a disease severity prediction or a disease progression.

12. The method of claim 9, further comprising determining, by the computing device, a treatment regimen based on the disease diagnosis.

13. The method of claim 9, wherein predicting the disease diagnosis comprises predicting the disease diagnosis based on a DNA methylation map and proximity of an active LINE-1 transposon to an oncogene associated with hepatocellular carcinoma (HCC).

14-26. (canceled)

27. A method for diagnosing or obtaining a prognosis for a cancer, the method comprising:
isolating or purifying deoxyribonucleic acids (DNA) from a patient sample;
bisulfite-treating the DNA;
preparing a sequencing library from the bisulfite-treated DNA;
hybridizing the DNA in the sequencing library with a probe panel to isolate a target gene from the DNA;
amplifying the target gene;
sequencing the target gene; and
diagnosing or obtaining a prognosis for the cancer.

28. (canceled)

29. The method of claim 27, wherein the probe panel targets virus integration sites, LINE-1 integration sites, introns and exons of oncogenes or tumor suppressors, methylation, or a combination thereof.

30. The method of claim 29, wherein the probe panel targets virus integration sites, LINE-1 integration sites, and methylation concurrently.

31-42. (canceled)

43. The method of claim 27, wherein:
the method further comprises sequencing the DNA in the sequencing library to generate sequence data; and
diagnosing or obtaining the prognosis for the cancer comprises:
identifying, by a computing device, hepatitis B virus (HBV) integration sites based on the sequence data;
identifying, by the computing device, active LINE-1 integration sites based on the sequence data;
identifying, by the computing device, hypomethylation sites based on the sequence data;
determining, by the computing device, a mutation analysis for an oncogene based on the sequence data; and
determining, by the computing device, a hepatocellular carcinoma (HCC) disease state progression based on the HBV integration sites, the active LINE-1 integration sites, the hypomethylation sites, and the mutation analysis.

44-49. (canceled)

50. A system for genomic analysis, the system comprising:
a sequencer to sequence a prepared biological sample from an individual to generate sequence data, wherein the prepared biological sample is bisulfate-treated and enriched with a hybridization probe panel that targets hepatitis B virus (HBV), LINE-1 transposon, and an oncogene associated with hepatocellular carcinoma (HCC); and
a computing device to:
identify HBV integration sites based on the sequence data;
identify active LINE-1 integration sites based on the sequence data;
identify hypomethylation sites based on the sequence data;
determine a mutation analysis for the oncogene based on the sequence data; and
determine an HCC disease state progression based on the HBV integration sites, the active LINE-1 integration sites, the hypomethylation sites, and the mutation analysis.

51-57. (canceled)

58. The system of claim 50, wherein the hybridization probe panel further targets a tumor suppressor gene associated with HCC.

59. The system of claim 58, wherein to determine the HCC disease state progression comprises to identify HBV integration sites, active LINE-1 integration sites, and hypomethylation sites in proximity to the oncogene or the tumor suppressor gene.

60. (canceled)
